(19) United States
(12) Patent Application Publication
(10) Pub. No.: US 2017/0354593 A1
(43) Pub. Date: Dec. 14, 2017

(54) PALATABLE CHEWABLE VETERINARY COMPOSITION

(71) Applicant: Zoetis Services LLC, Parsippany, NJ (US)

(72) Inventors: Kirit Majumdar, Turbhe (IN); Julie K. Lorenz, Kalamazoo, MI (US); Anjali Dandekar, Turbhe (IN)

(21) Appl. No.: 15/523,732

(22) PCT Filed: Nov. 2, 2015
(86) PCT No.: PCT/US2015/058602

§ 371 Date: May 2, 2017

Related U.S. Application Data
(60) Provisional application No. 62/074,275, filed on Nov. 3, 2014.

(51) Int. Cl. A61K 9/00; A61K 31/422; A61K 47/38; A61K 31/43; A61K 45/06; A61K 31/405; A61K 31/439; A61K 31/519; A61K 31/424

(52) U.S. Cl. CPC A61K 9/0056; A61K 31/519; A61K 31/422; A61K 47/38; A61K 31/424; A61K 45/06; A61K 31/405; A61K 31/439; A61K 31/43

(57) ABSTRACT

The present invention is directed to a palatable soft chew veterinary composition comprising at least one active agent, at least one binding agent, at least one disintegrant, at least one wetting agent, and at least one flavorant, and methods for controlling or treating a condition in an animal comprising administering the composition to said animal in need thereof.

### FIELD OF INVENTION

This invention relates to a palatable, soft chewable composition that comprises at least one active agent, wherein said agent is a systemically-acting agent. The composition is a veterinary composition for oral administration to animals.

### BACKGROUND OF INVENTION

Formulation of a drug (i.e., active agent) into an edible medication, such as a palatable, soft chewable dosage form, can increase subject acceptance of the medication, especially animals that tend to resist swallowing hard tablets or capsules and chewing bitter and/or granular dosage forms. Flavorings and polymeric coatings have commonly been used to provide some degree of palatability to the dosage form. Texture is also an issue for chewable medications. One of the most commonly used chewable dosage forms is the compressed tablet (i.e., hard chew), whose ingredients (including the actives and inactive ingredients such as binders and fillers) can make the tablet gritty or otherwise unappealing, especially to animals. Thus, a preferred alternative dosage form for use especially with animals is the palatable soft chew dosage form. This palatable soft chew dosage form is generally meat-like that is widely found in consumable pet treats, having a softness or palatability similar to cooked meat. Edible soft chews are typically manufactured by blending and extrusion, blending and knock-out, injection molds, and others. For extrusion, pre-mixed ingredients are introduced into an extruder barrel with a single or twin screw therein, then mixed, coagulated, expanded and sheared into a blended mixture, followed by application of additional heat or water for proper extrusion. The blended and extruded mixture is then formed into a desired shape on a die plate and cut into individual units. Consistency of texture, shape and weights of the chews from batch to batch of extruded material can also suffer. The additional heat and water can affect the stability of the active agent as well as alter the palatability of the composition. Further, it is highly desirable for the manufacturing means employed to produce soft chewable medications in an economical process that ensures composition stability, uniformity, and texture consistency, regardless of the active agent. The present invention achieves at least one of these economical outcomes.

### SUMMARY OF THE INVENTION

The present invention is directed to stable palatable, soft chewable compositions comprising at least one veterinary active agent. In accordance with this invention, it has been discovered that these compositions generally show increased stability of the active agents, regardless of the active agent concentrations and also palatability. Increased stability correlates with increased shelf-life, and optimally, efficacy while palatability increases patient compliance.

In one aspect of the invention, is a stable, palatable, soft chewable composition comprising a therapeutically effective amount of at least one active agent, at least one binding agent, at least one disintegrant, at least one wetting agent, and at least one flavorant.

In one aspect of the invention, the palatable, soft chewable composition comprises at least one active agent selected from the group consisting of an antiparasitic, anti-inflammatory, antipruritic, anti-emetic, and antibiotic agent.

In one aspect of the invention, the at least one active agent is an antiparasitic agent. In another aspect of the invention, the antiparasitic agent is an isoxazoline derivative. In another aspect of the invention, the isoxazoline derivative is selected from the group consisting of sarolaner, afoxolaner, fluralaner, and lotilaner. Sarolaner, the “S” enantiomer of 1-(5′-(5-(3,5-dichloro-4-fluorophenyl)-5-(trifluoromethyl)-4,5-dihydroisoxazol-3-yl)-3′H -spiro[azetidine-3,1′-isobenzofuran]-1-yl)-2-(methylsulfonyl)ethan-1-one, is the compound of Formula (A1),

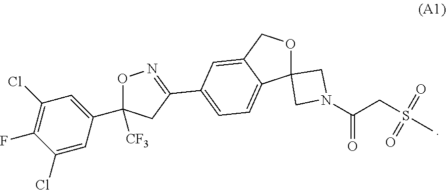 [SMILES: CS(=O)(=O)CC(=O)N1CC2(C1)OCc1cc(C3=NOC(c4cc(Cl)c(F)c(Cl)c4)(C(F)(F)F)C3)ccc12]

Afoxolaner, 4-(5-(3-chloro-5-(trifluoromethyl)phenyl)-5-(trifluoromethyl)-4,5-dihydroisoxazol-3-yl) -N-(2-oxo-2-((2,2,2-trifluoroethyl)amino)ethyl)-1-naphthamide, and stereoisomers thereof, is the compound of Formula (A2)

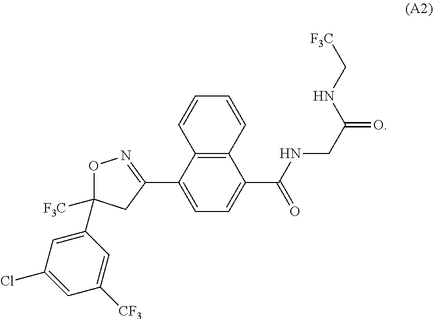 [SMILES: O=C(CNC(=O)c1ccc(C2=NOC(c3cc(Cl)cc(C(F)(F)F)c3)(C(F)(F)F)C2)c2ccccc12)NCC(F)(F)F]

Fluralaner, 4-(5-(3,5-dichlorophenyl)-5-(trifluoromethyl)-4,5-dihydroisoxazol-3-yl) -2-methyl-N-(2-oxo-2-((2,2,2-trifluoroethyl)amino)ethyl)benzamide, and stereoisomers thereof, is a compound of Formula (A3)

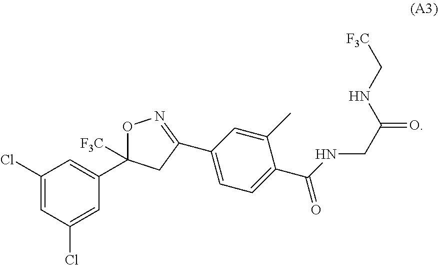 [SMILES: Cc1cc(C2=NOC(c3cc(Cl)cc(Cl)c3)(C(F)(F)F)C2)ccc1C(=O)NCC(=O)NCC(F)(F)F]

Lotilaner, 3-methyl-N-{2-oxo-2-[(2,2,2-trifluoroethyl)amino]ethyl}-5-[(5S)-5-(3,4,5-trichlorophenyl)-5-(trifluoromethyl) -4,5-dihydro-1,2-oxazol-3-yl]thiophene-2-carboxamide, and stereoisomers thereof, is a compound of Formula (A4)

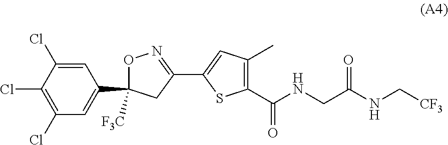 [SMILES: Cc1cc(C2=NO[C@@](c3cc(Cl)c(Cl)c(Cl)c3)(C(F)(F)F)C2)sc1C(=O)NCC(=O)NCC(F)(F)F]

In another aspect of the invention, the antiparasitic agent, a compound of Formula (A1), (A2), (A3), or (A4) can be further combined with at least one additional antiparasitic agent. The at least one additional antiparasitic agent is selected from the group consisting of a macrocyclic lactone (e.g., moxidectin, ivermectin, selamectin, dimadectin, eprinomectin, abamectin, doramectin, emamectin, latidectin, lepimectin, milbemycin and milbemycin oxime), pyrantel, including the salt forms (e.g., pamoate, citrate, and tartrate), lufenuron, emodepside, oxfendazole, albendazole, triclabendazole, levamisole, closantel, clorsulon, praziquantel, and mixtures thereof. In yet another aspect of the invention, is a palatable, soft chewable composition comprising a compound of Formula (1A) and at least one additional antiparasitic agent is selected from the group consisting of a macrocyclic lactone (e.g., moxidectin, ivermectin, selamectin, dimadectin, eprinomectin, abamectin, doramectin, emamectin, latidectin, lepimectin, milbemycin, and milbemycin oxime), pyrantel, including the salt forms (e.g., pamoate, citrate, and tartrate), lufenuron, oxfendazole, albendazole, triclabendazole, levamisole, closantel, clorsulon, praziquantel, and mixtures thereof. In yet another aspect of the invention, is a palatable, soft chewable composition comprising a compound of Formula (1A) and at least one additional antiparasitic agent is selected from the group consisting of a macrocyclic lactone (e.g., moxidectin, ivermectin, eprinomectin, abamectin, doramectin, milbemycin, and milbemycin oxime), pyrantel, including the salt forms (e.g., pamoate, citrate, and tartrate), triclabendazole, levamisole, closantel, clorsulon, praziquantel, and mixtures thereof. In yet another aspect of the invention, is a palatable, soft chewable composition comprising a compound of Formula (1A) and at least one additional antiparasitic agent is selected from the group consisting of moxidectin, doramectin, ivermectin, abamectin, milbemycin, milbemycin oxime, pyrantel, including the salt forms (e.g., pamoate, citrate, and tartrate), levamisole, praziquantel, and mixtures thereof. In yet another aspect of the invention, is a palatable, soft chewable composition comprising a compound of Formula (1A) and moxidectin. In yet another aspect of the invention, is a soft chewable composition comprising a compound of Formula (1A), moxidectin and pyrantel. In yet another aspect of the invention, is a soft chewable composition comprising a compound of Formula (1A) and milbemycin oxime. In yet another aspect of the invention, is a soft chewable composition comprising a compound of Formula (1A), milbemycin oxime, and pyrantel.

In another aspect of the invention, is a stable, palatable, soft chewable composition comprising at least one active agent, wherein the one active agent is the anti-inflammatory agent, carprofen, 2-(6-chloro-9H-carbazol-2-yl)propanoic acid, and stereoisomers thereof, that is a compound of Formula (B)

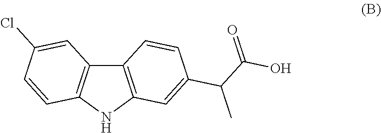 [SMILES: CC(C(=O)O)c1ccc2c(c1)[nH]c1ccc(Cl)cc12]

In one aspect of the invention, is a stable, palatable, soft chewable composition comprising at least one active agent, wherein the active agent is the anti-pruritic agent, oclacitinib, N-methyl-1-(4-(methyl(7H-pyrrolo[2,3-d]pyrimidin-4-yl)amino)cyclohexyl)methanesulfonamide, and in particular, N-methyl-[trans-4-(methyl-7H-pyrrolo[2,3-d]pyrimidin-4-ylamino)cyclohexyl]methanesulfonamide, and the maleate salt, particularly, (2Z)-2-butenedioate, that is a compound of Formula (C). Oclacitinib maleate is Apoquel®.

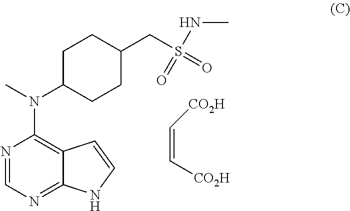

In yet another aspect of the invention, is a stable, palatable, soft chewable composition comprising at least one active agent, wherein the active agent is the anti-emetic agent, maropitant, (2S,3S)-N-(5-(tert-butyl-2-methoxyphenyl)-methyl-2-diphenylmethyl-1-azabicyclo[2.2.2]octan-3-amine, particularly the citrate and citrate monohydrate salts, that is a compound of Formula (D). Maropitant citrate monohydrate is Cerenia®.

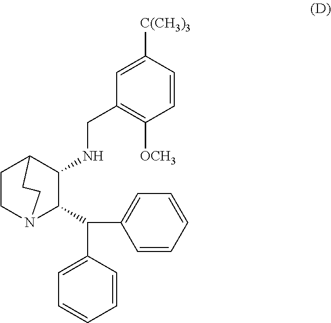 [SMILES: COc1ccc(C(C)(C)C)cc1CN[C@H]1C2CCN(CC2)[C@H]1C(c1ccccc1)c1ccccc1]

In yet another aspect of the invention, is a palatable, soft chewable composition comprising at least one active agent, wherein the active agent is an antibiotic agent. In another aspect of the invention, the antibiotic agent is selected from the group consisting of cefpodoxime proxetil (Simplicef®), clavulanic acid and amoxicillin (Clavamox®), and doxycycline hyclate (e.g., Vibramycin®, Doryx®, Adoxa®, and the like).

In one aspect of the invention is a palatable, soft chewable composition where in the at least one binding agent, is a veterinary acceptable binding agent. In one aspect the veterinary acceptable binding agent is selected from the group consisting of microcrystalline cellulose, hydroxypropyl cellulose, hydroxypropyl methyl cellulose, ethyl cellulose, polyvinylpyrrolidone (e.g., povidone (Kollidon 25, 30, and 90) and co-povidone (Kollidon VA 64), polyethylene glycol, acacia, tragacanth gum, gelatin, sucrose, lactose (e.g., hydrous, anhydrous, monohydrate), xylitol, sorbitol, maltitol, corn starch, potato starch, carnauba wax, alginate, and mixtures thereof. In yet another aspect of the invention, the binding agent is selected from the group consisting of polyvinylpyrrolidone, microcrystalline cellulose, hydroxypropyl methyl cellulose, and mixtures thereof. In yet another aspect of the invention, the binding agent is selected from the group consisting of polyvinylpyrrolidone and microcrystalline cellulose, and mixtures thereof.

In one aspect of the invention, is a palatable, soft chewable composition wherein the at least one veterinary acceptable disintegrant is selected from the group consisting of croscarmellose sodium, citric acid, and sodium starch glycolate, and mixtures thereof. In yet another aspect of the invention, the at least one disintegrant is croscarmellose sodium. In yet another aspect of the invention, the at least one disintegrant is citric acid. In yet another aspect of the invention, the at least one disintegrant is sodium starch glycolate.

In one aspect of the invention is a palatable, soft chewable composition wherein the at least one veterinary acceptable wetting agent is selected from the group consisting of hydrous and anhydrous solvents. Non-limiting examples of wetting agents include: water, glycerin, propylene glycol, polyethylene glycol, ethanol, polysorbate 80, triacetin, and mixtures thereof. In yet another aspect of the invention, the wetting agent is glycerin. In yet another aspect of the invention, the wetting agent is glycerin and water. In yet another aspect of the invention, the wetting agent is propylene glycol and glycerin.

In one aspect of the invention is a palatable, soft chewable composition, wherein the at least one veterinary acceptable flavorant is an artificial flavorant, natural flavorant, and mixture thereof. In yet another aspect of the invention, the artificial flavorant is selected from at least one of chicken, turkey, beef, pork, lamb, and fish. In one aspect of the invention, the natural flavorant is selected from at least one of chicken, turkey, beef, pork, lamb, and fish, which can be obtained from meat, meat products, organ meat (e.g., liver, kidney, and the like), and mixtures thereof. Flavorants can also include vegetable matter and yeast extracts, for example brewer's yeast and hydrolyzed vegetable protein. In yet another aspect of the invention, the palatable soft chew composition further comprises at least one veterinary acceptable flavor enhancer.

In yet another aspect of the invention, the palatable, soft chewable composition further comprises at least one veterinary acceptable antioxidant, flavor enhancer, and mixture thereof.

In yet another aspect of the invention, the palatable, soft chewable composition further comprises at least one veterinary acceptable excipient.

In yet another aspect of the invention is a palatable, soft chewable composition for manufacture of a medicament. In another aspect of the invention, the medicament is orally administered to an animal in need thereof.

In yet another aspect of the invention is a method for treating a parasitic infection or infestation in an animal, by administering a composition of the invention, wherein said composition comprises at least one veterinary antiparasitic agent, to the animal in need thereof. In another aspect of the invention is a method for treating a parasitic infection or infestation in an animal, by administering a composition of the invention, wherein said composition comprises at least one veterinary antiparasitic agent of Formula (A1), (A2), (A3), or (A4) to the animal in need thereof. In another aspect of the invention is a method for treating a parasitic infection or infestation in an animal, by administering a composition of the invention, wherein said composition comprises a veterinary acceptable antiparasitic agent that is the compound of Formula (A1). In another aspect of the invention is a method for treating a parasitic infection or infestation in an animal, by administering a composition of the invention, wherein said composition comprises the veterinary acceptable antiparasitic agent that is the compound of Formula (A1) and further comprising at least one additional antiparasitic agent. In another aspect of the invention is a method for treating a parasitic infection or infestation in an animal, by administering a composition of the invention, wherein said composition comprises the veterinary acceptable antiparasitic agent that is the compound of Formula (A1) and further comprises at least one additional veterinary acceptable agent selected from moxidectin, milbemycin, pyrantel, and mixtures thereof. In another aspect of the invention is a method for treating a parasitic infection or infestation in an animal, by administering a composition of the invention, wherein said composition comprises the veterinary acceptable antiparasitic agent that is the compound of Formula (A1) and further comprises at least one additional veterinary acceptable agent selected from moxidectin and pyrantel, and mixtures thereof.

In another aspect of the invention is a method for treating a parasitic infection or infestation in an animal, by administering a composition of the invention, wherein said composition comprises a veterinary acceptable antiparasitic agent that is the compound of Formula (A2), (A3), or (A4). In another aspect of the invention is a method for treating a parasitic infection or infestation in an animal, by administering a composition of the invention, wherein said composition comprises the veterinary acceptable antiparasitic agent that is the compound of Formula (A2), (A3), or (A4) and further comprising at least one additional antiparasitic agent.

In yet another aspect of the invention is a method for treating emesis in an animal by administering a composition of the present invention, wherein said composition comprises maropitant.

In yet another aspect of the invention is a method for treating pruritis and atopic dermatitis in an animal, by administering a composition of the present invention, wherein said composition comprises oclacitinib.

In yet another aspect of the invention is a method for treating pain and inflammation in an animal, by administering a composition of the present invention, wherein said composition comprises carprofen.

In yet another aspect of the invention, the palatable soft chew composition is administered orally.

### DEFINITIONS

For purposes of the present invention, as described and claimed herein, the following terms and phrases are defined as follows:

“Additional antiparasitic agent(s)” as used herein, unless otherwise indicated, refers to at least one additional veterinary or pharmaceutical compound or product that provides a therapeutically effective amount of compound or product that is useful for the treatment of a parasitic infection or infestation in or on an animal

“Animal”, as used herein, unless otherwise indicated, refers to an individual animal that is a mammal. Specifically, mammal refers to a vertebrate animal that is human and non-human, which are members of the taxonomic class Mammalia. Non-exclusive examples of non-human mammals include companion animals and livestock. Non-exclusive examples of a companion animal include: dog, cat, and horse. Preferred companion animals are dog and cat. More preferred is dog. Non-exclusive examples of livestock include: pig, llama, rabbits, goat, sheep, deer, elk, and cattle.

“Infection” or Infestation”, as used herein, unless otherwise indicated, refers to the state or condition of having parasites on or in the body.

“Macrocyclic lactone” as used herein, designates a pharmaceutical or veterinary compound in the avermectin family of compounds including, for example, ivermectin, abamectin doramectin, eprinomectin, selamectin, and the like; and also the milbemycin family of compounds including, for example, moxidectin, milbemycin, milbemycin oxime, and the like.

“Parasite(s)”, as used herein, unless otherwise indicated, refers to endoparasites and ectoparasites. Endoparasites are parasites that live within the body of its host and include helminths (e.g., trematodes, cestodes, and nematodes) and protozoa. Ectoparasites are organisms of the Arthropoda phylum (e.g., arachnids and insects) which feed through or upon the skin of its host. Preferred arachnids are of the order Acarina, e.g., ticks and mites. Preferred insects are midges, fleas, mosquitoes, biting flies (stable fly, horn fly, sand fly, blow fly, horse fly, and the like), bed bugs, and lice. Preferred compositions of the present invention can be used for the treatment of parasites, i.e., treatment of a parasitic infection or infestation. Parasite(s) also encompasses the different life stages of the ectoparasite and endoparasite, including eggs, pupae, and larvae which feed on or in the body.

“Soft chew”, as used herein, unless otherwise indicated, refers to a ductile composition, wherein ductile simply refers to a supple, pliable, flexible form, i.e., is not brittle.

“Therapeutically effective amount”, as used herein, unless otherwise indicated, refers to an amount of one active agent or combination of active agents that treats, prevents, attenuates, ameliorates, delays, or eliminates one or more symptoms, for example: (i) the particular parasitic infection or infestation for an antiparasitic agent (ii) emesis, pruritis, and/or visceral pain for maropitant, (iii) the particular infection for an antibiotic agent, (iv) inflammation and pain for a non-steroidal anti-inflammatory drug, for example carprofen, ketoprofen, and prodrugs of ketoprofen, (v) atopic dermatitis for a JAK inhibitor, for example apoquel.

“Treatment”, “treating”, and the like, as used herein, unless otherwise indicated, refers to reversing, alleviating, or inhibiting the parasitic or bacterial infection, emesis, visceral pain, and pruritis. As used herein, these terms also encompass, depending on the condition of the animal, preventing the onset of a disorder or condition, or of symptoms associated with a disorder or condition, including reducing the severity of a disorder or condition or symptoms associated therewith prior to affliction with said infection, infestation, or condition. Thus, treatment can refer to administration of the composition with at least one veterinary agent to the animal that is not at the time of administration afflicted with said condition. Treating also encompasses preventing the recurrence of an infection or infestation or of symptoms associated therewith as well as references to “control” (e.g., kill, repel, expel, incapacitate, deter, eliminate, alleviate, minimize, and eradicate).

“Veterinary acceptable” as used herein, unless otherwise indicated, indicates that the substance or composition must be compatible chemically and/or toxicologically, with the other ingredients comprising a formulation, composition, and/or the animal being treated therewith. The term “pharmaceutically” acceptable has the same meaning as that recited for “veterinary” acceptable.

As used herein, percent of components of the soft chew means and refers to percentages of the total weight of the soft chew.

“% w/w” or “w/w %” herein refers to the mass fraction of a given composition component expressed as a percentage, determined according to the formula mᵢ/mₜₒₜ×100, wherein mᵢ is the mass of the substance of interest present in the composition, and mₜₒₜ is the total mass of the composition.

### DETAILED DESCRIPTION

The present invention provides novel and inventive palatable, soft chewable compositions for oral administration of a wide variety of veterinary acceptable active agents. The palatable, soft chew composition of the present invention comprises at least one veterinary acceptable active agent, at least one veterinary acceptable binding agent, at least one veterinary acceptable disintegrant, at least one veterinary acceptable wetting agent, and at least one veterinary acceptable flavorant. The palatable, soft chew composition of the present invention comprises at least one veterinary acceptable active agent, at least one veterinary acceptable binding agent, at least one veterinary acceptable disintegrant, at least one veterinary acceptable wetting agent, at least one veterinary acceptable flavorant, and further comprises at least one veterinary acceptable flavor enhancer, antioxidant, excipient, filler, and mixtures thereof.

Suitable veterinary acceptable active agents include pharmaceuticals, minerals, vitamins, and other nutraceuticals. Suitable non-limiting active agents include analgesics (for example, non-steroidal anti-inflammatory drugs (e.g, carprofen, flunixine meglumine, ketoprofen, ketoprofen methyl ester, naproxen, meloxicam, robenacoxib, and the like) and others, for example, medetomidine, phenylbutazone, hydromorphone, and the like); anti-emetics (e.g., maropitant, and maropitant salts, dextromethorphan, diphenhydramine, 8-chlorotheophylline, cisapride, omeprazol, famotidine, metoclopramide, promethazine, dolasetron, ondansetron, granisetron, ketamine, lansoprasol, meclizine, mirtazepine, and the like); antihistamines/antipyretics (e.g., acepromazine, clemastine fumarate, cyproheptadine, famotidine, loratadine, hydroxyzine, meclizine hydrochloride, apoquel, chlorpheniramine, diphenhydramine, and the like); antiparasitics (e.g., macrocyclic lactones (ivermectin, abamectin, doramectin, emamectin, moxidectin, milbemycin, milbemycin oxime, and the like), imidacloprid, emodepside, levamisole, pyrantel, pyrantel pamoate, isoxazolines (e.g., sarolaner, afoxolaner, lotilaner, and fluralaner), derquantel, anticoccidials, benzimidazoles (thiabendazole, mebendazole, fenbendazole, oxfendazole, albendazole, and the like), antimicrobials (e.g., pleuromutilins, polymyxins, aminoglycosides, fluoroquinolones (e.g., danofloxacin, ciprofloxacin, norfloxacin, ofloxacin, levofloxacin, and the like), macrolides (e.g., azithromycin, erythromycin, telithromycin and the like), lincosamides (e.g., clindamycin), aminoglycosides (e.g., amikacin, streptomycin, tobramycin, and the like), sulfonamides (e.g., sulfadoxine, sulfamethizole, sulfisoxazole, and the like), penicillins, beta-lactams, tetracyclines (e.g., doxycycline hyclate, minocycline, and the like), aminopenicillins, cephalosporins (1^{st}-4^{th} generations, e.g., simplicef, ceftiofur, cefovecin, and the like), and the like; and mixtures thereof. Preferred active agents include apoquel, carprofen, sarolaner, afoxolaner, fluralaner, lotilaner, maropitant, acetaminophen, ibuprofen, flurbiprofen, clavamox, naproxen, meloxicam, ketoprofen, phenylpropanolamine, chlorpheniramine maleate, dextromethorphan, diphenhydramine, famotidine, loperamide, ranitidine, cimetidine, astemizole, terfenadine, terfenadine carboxylate, cetirizine, mixtures thereof, and pharmaceutically acceptable salts thereof. More preferred active agents include sarolaner, maropitant, apoquel, and carprofen.

A suitable veterinary acceptable active agent is sarolaner, the “S” enantiomer of 1-(5′-(5-(3,5-dichloro-4-fluorophenyl)-5-(trifluoromethyl)-4,5-dihydroisoxazol-3-yl) -3′H-spiro[azetidine-3,1′-isobenzofuran]-1-yl)-2-(methylsulfonyl)ethan-1-one, a compound of Formula (A1),

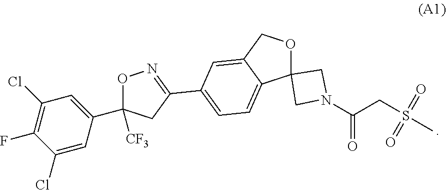 [SMILES: CS(=O)(=O)CC(=O)N1CC2(C1)OCc1cc(C3=NOC(c4cc(Cl)c(F)c(Cl)c4)(C(F)(F)F)C3)ccc12]

A suitable veterinary acceptable active agent is afoxolaner, 4-(5-(3-chloro-5-(trifluoromethyl)phenyl)-5-(trifluoromethyl)-4,5-dihydroisoxazol-3-yl) -N-(2-oxo-2-((2,2,2-trifluoroethyl)amino)ethyl)-1-naphthamide, and stereoisomers thereof, a compound of Formula (A2)

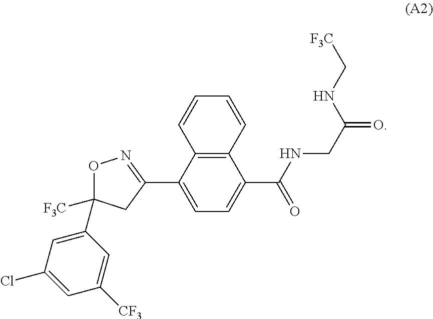 [SMILES: O=C(CNC(=O)c1ccc(C2=NOC(c3cc(Cl)cc(C(F)(F)F)c3)(C(F)(F)F)C2)c2ccccc12)NCC(F)(F)F]

A suitable veterinary acceptable active agent is fluralaner, 4-(5-(3,5-dichlorophenyl)-5-(trifluoromethyl)-4,5-dihydroisoxazol-3-yl) -2-methyl-N-(2-oxo-2-((2,2,2-trifluoroethyl)amino)ethyl)-benzamide, and stereoisomers thereof, a compound of Formula (A3)

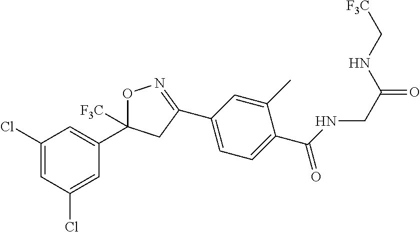 [SMILES: Cc1cc(C2=NOC(c3cc(Cl)cc(Cl)c3)(C(F)(F)F)C2)ccc1C(=O)NCC(=O)NCC(F)(F)F]

A suitable veterinary acceptable active agent is lotilaner, 3-methyl-N-{2-oxo-2-[(2,2,2-trifluoroethyl)amino]ethyl}-5-[(5S)-5-(3,4,5-trichlorophenyl)-5-(trifluoromethyl)-4,5-dihydro-1,2-oxazol-3-yl]thiophene-2-carboxamide, and stereoisomers thereof, a compound of Formula (A4)

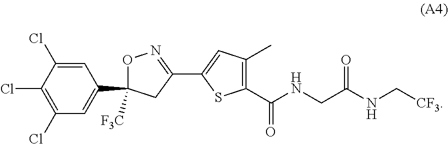 [SMILES: Cc1cc(C2=NO[C@@](c3cc(Cl)c(Cl)c(Cl)c3)(C(F)(F)F)C2)sc1C(=O)NCC(=O)NCC(F)(F)F]

The palatable soft chew composition comprising at least one antiparasitic agent selected from a compound of Formula (A1), (A2), (A3), or (A4) further comprises at least one additional antiparasitic agent. The at least one additional antiparasitic agent is selected from the group consisting of a macrocyclic lactone (e.g., moxidectin, ivermectin, selamectin, dimadectin, eprinomectin, abamectin, doramectin, emamectin, latidectin, and lepimectin), milbemycin, milbemycin D, milbemycin A₃, milbemycin A₄, milbemycin oxime, pyrantel, including the salt forms (e.g., pamoate, citrate, and tartrate), insect growth regulators (IGR's) (including juvenile hormone mimics and chitin synthesis inhibitors), for example: azadirachtin, diofenolan, fenoxycarb, hydroprene, kinoprene, methoprene, pyriproxyfen, tetrahydroazadirachtin, 4-chloro-2(2-chloro-2-methyl-propyl)-5-(6-iodo-3-pyridylmethoxy)pyridazine-3(2H)-one, chlorofluazuron, cyromazine, diflubenzuron, fluazuron, flucycloxuron, flufenoxuron, hexaflumoron, lufenuron, tebufenozide, teflubenzuron, triflumoron, 1-(2,6-difluorobenzoyI)-3-(2-fluoro-4-(trifluoromethyl)phenylurea, 1-(2,6-difluoro-benzoyl)-3-(2-fluoro-4-(1,1,2,2-tetrafluoroethoxy)-phenylurea and 1-(2,6-difluorobenzoyI)-3-(2-fluoro-4-trifluoromethyl)phenylurea; benzimidazole agents (e.g., thiabendazole, oxibendazole, mebendazole, fenbendazole, oxfendazole, albendazole, and triclabendazole), febantel, levamisole, morantel, praziquantel, closantel, clorsulon, and amino acetonitrile active agents, and combinations thereof. The preferred palatable soft chewable composition comprises the compound of Formula (1A) and further comprises moxidectin. Another preferred palatable soft chewable composition comprises the compound of Formula (1A) and further comprises moxidectin and pyrantel. Another preferred palatable soft chewable composition comprises the compound of Formula (1A) and further comprises milbemycin oxime. Another preferred palatable soft chewable composition comprises the compound of Formula (1A) and further comprises milbemycin oxime and pyrantel.

The palatable soft chew composition comprises the active agent, carprofen, 2-(6-chloro-9H-carbazol-2-yl) propanoic acid, and stereoisomers thereof, that is a compound of Formula (B)

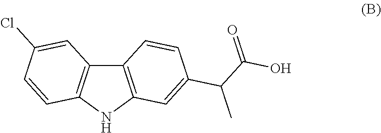 [SMILES: CC(C(=O)O)c1ccc2c(c1)[nH]c1ccc(Cl)cc12]

is the preferred anti-inflammatory agent.

The palatable soft chew composition comprises the active agent, oclacitinib (apoquel, the maleate salt of oclacitinib), N-methyl-1-(4-(methyl(7H-pyrrolo[2,3-d]pyrimidin-4-yl)amino)cyclohexyl)methanesulfonamide, and in particular, N-methyl-[trans-4-(methyl-7H-pyrrolo[2,3-d]pyrimidin-4-ylamino)cyclohexyl]methanesulfonamide, and the maleate salt, particularly, (2Z)-2-butenedioate, that is a compound of Formula (C),

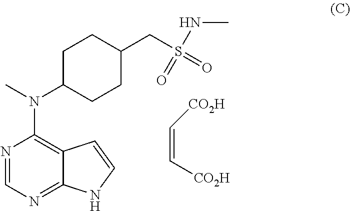

is the preferred anti-pruritic agent.

The palatable soft chew composition comprises the active agent, maropitant (cerenia), (2S,3S)-N-(5-(tert-butyl-2-methoxyphenyl)-methyl-2-diphenylmethyl-1-azabicyclo[2.2.2]octan-3-amine, particularly the citrate and citrate monohydrate salts, that is a compound of Formula (D)

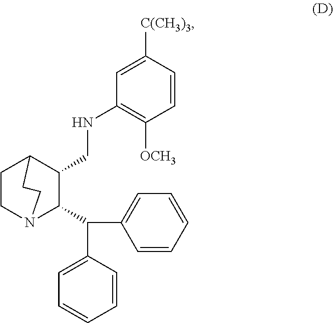 [SMILES: COc1ccc(C(C)(C)C)cc1NC[C@H]1C2CCN(CC2)[C@H]1C(c1ccccc1)c1ccccc1]

is the preferred anti-emetic.

The palatable soft chew composition comprises an antibacterial as the active agent. For example, the antibiotic agent is selected from the group consisting of clavulanic acid and amoxicillin (Clavamox®), cefpodoxime proxetil, and doxycycline hyclate.

The active agent(s) are present in the tablet in a therapeutically effective amount, which is an amount that produces the desired therapeutic response upon oral administration and can be readily determined by one skilled in the art. In determining such amounts, the particular compound being administered, the bioavailability characteristics of the ingredient, the dose regime, the age and weight of the patient, and other factors must be considered. For example, carprofen is administered as a 25, 75, and 100 mg tablet to provide a dose of about 2 mg/pound. In contrast, maropitant tablets are prepared as a 16 mg, 24 mg, 60 mg, and 160 mg tablet to provide an 8 mg/kg dose, depending on the size of the animal.

The veterinary acceptable binding agent in the present invention is used to add cohesiveness to the formulation, thereby providing the necessary bonding to form a cohesive mass and to ensure a suitable compacted tablet form. These binding agents are conventionally used in direct compression tablets and are described in Lieberman et. al., Pharmaceutical Dosage Forms, 2 Ed. , Vol. 1, pp. 209-214 (1990). These binding agents can also be used in the preparation of soft chew tablets that are prepared from extrusion processes. Non-limiting examples of veterinary acceptable binding agents include: microcrystalline cellulose, hydroxypropyl cellulose, hydroxypropyl methyl cellulose, ethyl cellulose, polyvinylpyrrolidone (e.g., povidone (Kollidon 25, 30, and 90) and co-povidone (Kollidon VA 64), polyethylene glycol, acacia, tragacanth gum, gelatin, sucrose, lactose, xylitol, sorbitol, maltitol, corn starch, potato starch, carnauba wax, alginate, and mixtures thereof. The preferred binding agents include: polyvinylpyrrolidone, microcrystalline cellulose, hydroxypropyl methyl cellulose, and mixtures thereof. The more preferred binding agents include: polyvinylpyrrolidone and microcrystalline cellulose, and mixtures thereof. The binding agent(s) are typically present in the palatable soft chew composition at a concentration of about 10% w/w to about 25% w/w. More typically, the binding agent(s) may be present at concentrations of about 12% w/w to about 20% w/w, with a preferred concentration of about 14% w/w to about 18% w/w, based on total weight of the soft chew.

The soft chewable composition comprises at least one veterinary acceptable disintegrant, thereby providing a means for the dosage form to expand and dissolve more readily when wet. Disintegrants are conventionally used in direct compression tablets and are described in Lieberman et al., Pharmaceutical Dosage Forms, 2 Ed., Vol. 1, pp. 209-214 (1990). Disintegrants are used in the preparation of soft chew tablets that are prepared from extrusion processes. Non-exclusive examples of veterinary acceptable disintegrants include: cellulose, carboxymethyl cellulose calcium, carboxymethyl cellulose sodium, hydroxypropyl starch, lactose monohydrate, hydroxypropyl cellulose, crospovidone, magnesium aluminum silicate, guar gum, alginic acid, sodium alginate, calcium alginate, chitosan, croscarmellose sodium (e.g., Ac-Di-Sol®), citric acid, sodium starch glycolate, and the like, and mixtures thereof. The preferred disintegrant(s) is selected from the group consisting of carboxymethyl cellulose calcium, carboxymethyl cellulose sodium, hydroxypropyl cellulose, crospovidone, croscarmellose sodium, and sodium starch glycolate, and mixtures thereof. The more preferred disintegrant is croscarmellose sodium. Typically, the disintegrant(s) in the palatable soft chew composition is at a concentration of about 3% w/w to about 14% w/w. More typically, the concentration of the disintegrant(s) is about 4% w/w to about 12% w/w. Even more typically, the concentration of the disintegrant(s) is about 5% w/w to about 12% w/w, based on total weight of the soft chew.

The soft chewable composition comprises at least one veterinary acceptable wetting agent (i.e., humectant). The humectant is selected from hydrous and anhydrous solvents. Non-exclusive examples of veterinary acceptable wetting agents include: mineral oil, glycerin, glycerol formal, miglyol (e.g., miglyol 812, miglyol 840), Solutol HS 15 (polyglycol mono- and di-esters of 12-hydroxystearic acid), ethylene glycol, propylene glycol, methoxypropanol, diethylene glycol monoethyl ether, diethylene glycol monomethyl ether, diethylene glycol monobutyl ether, tetraglycol, triethylene glycol, butyl diglycol, dimethylacetamide, dimethylformamide, n-methylformamide, dipropylene glycol n-butyl ether, ethanol, isopropanol, methanol, ethylene glycol monoethyl ether, ethylene glycol monomethyl ether, dipropyleneglycol monomethyl ether, dipropyleneglycol monomethyl ether, dipropyleneglycol monomethyl ether, triethylene glycol monoethyl ether, triethylene glycol monomethyl ether, polyethylene glycols, methoxypolyethylene glycols, polypropylene glycols, polybutylene glycols, diethylene monoethylether acetate, diethylene monobutylether acetate, monomethylacetamide, liquid polyoxyethylene glycols, 2-pyrrolidone, propylene carbonate, butylene carbonate, tetrahydrofurfuryl alcohol, solketal, xylene, dimethyl isosorbide, short-, medium- and long chain, and aromatic fatty acids, for example butyric acid, capric acid, succinic, adipic, sebacic, capriylic acid, lauric acid, myristic acid, strearic acid, linoleic acid, and benzoic acid, triglycerides, for example, castor oil, cottonseed oil, sesame oil, linseed oil, safflower oil, peanut oil, soybean oil, coconut oil, olive oil, corn oil, and almond oil. The veterinary acceptable wetting agents can also include: glyceryl stearates, glyceryl hexanoates, caprylic/capric glycerides, triacetin, glyceryl cocoate, caprylic glycerides, glyceryl, glyceryl stearates, glyceryl hexanoates, glyceryl monooleate, glyceryl ricinoleate, capric glycerides, succinic acid, isopropyl myristate, ethyl oleate, ethyl laurate, dibutyl adipate, propylene glycol monocaprylate, propylene glycol monolaurate, spider esters, dibutyl sebacate, dibutyl adipate, 2-pyrrolidone, and N-methyl pyrrolidone. Preferred wetting agents include: glycerin, miglyol, Solutol HS 15 (polyglycol mono- and di-esters of 12-hydroxystearic acid), propylene glycol, ethanol, polypropylene glycols, triglycerides, for example, castor oil, cottonseed oil, sesame oil, safflower oil, peanut oil, soybean oil, coconut oil, and olive oil. The more preferred wetting agents include: glycerin, miglyol, propylene glycol, ethanol, polypropylene glycols, castor oil, cottonseed oil, sesame oil, safflower oil, soybean oil, and olive oil. Even more preferred, the wetting agent is glycerin. Typically, wetting agent(s) are present in the palatable soft chew composition at concentrations of about 15% w/w to about 40% w/w. More typically, the wetting agent(s) are present at concentrations of about 20% w/w to about 35% w/w. Even more typically, the wetting agent(s) are present at concentrations of about 25% w/w to about 30% w/w, based on total weight of the soft chew. It is preferable for the wetting agent to be selected from the group consisting of glycerol, propylene glycol, and mixtures thereof, with glycerol being most preferred. The amount of wetting agent in the composition is chosen so that the product body remains soft.

The palatable soft chewable composition comprises at least one flavorant. Flavorants are used to alter or enhance the flavor(s) of natural food product such as meats and vegetables, or creating additional flavor for food products that do not have the desired flavors such as snacks and oral medications. Most types of flavorants are focused on scent and taste. Artificial flavorants are chemically synthesized compounds that are used to flavor food items and are often formulated with the same chemical compounds found in natural flavorants. Most artificial flavors are specific and often complex mixtures of singular naturally occurring flavor compounds combined together to either imitate or enhance a natural flavor. These mixtures are formulated by flavorists to give a food product a unique flavor and to maintain flavor consistency between different product batches or after recipe changes. The list of known flavoring agents includes thousands of molecular compounds, and can be combined to achieve flavors similar to chicken, turkey, beef, pork, lamb, fish, egg, cheese, seafood, smoke, and mixtures thereof. A natural flavorant is defined as the essential oil, oleoresin, essence or extractive, protein hydrolysate, distillate, or any product of roasting, heating or enzymolysis, which contains the flavoring constituents derived from a spice, fruit or fruit juice, vegetable or vegetable juice, edible yeast, herb, bark, bud, root, leaf or any other edible portions of a plant, meat, seafood, poultry, eggs, dairy products, or fermentation products thereof, whose primary function in food is flavoring rather than nutritional. Natural flavorants include chicken, turkey, beef, pork, lamb, fish, egg, cheese, seafood, vegetable, and mixtures thereof. Yeast extracts are also included in the natural flavors. Natural meat flavorants can be obtained from meat, meat products, organ meat, yeast extracts, vegetable matter, and mixtures thereof. For example, an oral veterinary composition medication might include animal product-based flavorings such as dried or powdered meat and meat parts such as beef, pork, chicken, turkey, fish, and lamb; organ meats such as liver; meat meals, bone meals and ground bone; and animal-derived food such as casein, milk (which may include dry forms and lowered fat forms, such as dry skim milk), yogurt, gelatin, cheese and egg (collectively, “animal origin flavorings”) may be utilized. The flavorant can be added directly to the composition as part of the dry blend or it can be added to other dry ingredients (fillers) to prepare a flavorant admixture to be added to the dry blend. Various fillers known in the art may be used in the palatable soft chewable composition. Non-limiting examples of fillers include: starch (e.g., corn, potato, tapioca, and the like), sugars (e.g., sucrose, fructose, lactose, mannitol, and the like, including hydrous and anhydrous forms), gelatin, cellulose (e.g., methyl cellulose, ethyl cellulose, and the like), calcium phosphate, soy protein fines, corn cob, corn gluten meal, and the like, and mixtures thereof. The flavorant/filler admixture typically contains at least one flavorant at a concentration of about 10% w/w to about 40% w/w, based on total weight of the admixture. More typically, the flavorant(s) is at a concentration of about 15% w/w to about 30% w/w, based on total weight of the admixture.

The palatable soft chew composition can further comprise active agents that have been made more palatable via taste masking techniques. These methods include coating, granulation, or complexing to carriers as examples.

Taste masking compositions suitable for use in the present invention include, but are not limited to cellulose acetate, cellulose acetate butyrate, Eudragit E100, ethyl cellulose, sodium chloride, polyvinylpyrrolidones (e.g., K12, K17, K25, K30, and K90), hydroxypropyl methylcellulose, hydroxypropylcellulose, 2-vinyl pyrridine styrene, cellulose triacetate, and the use of polymer-based technologies can be used for taste masking.

The palatable soft chew composition further comprises at least one anti-oxidant. Non-exclusive examples of antioxidants include: ascorbic acid, vitamin E (tocopherol), vitamin E derivatives, sodium metabisulphate, ascrobyl palmitate, fumaric acid, malic acid, sodium ascorbate, butylated hydroxanisole (BHA), and butylated hydroxytoluene (BHT), citric acid, propyl gallate, thioglycerol, and the like, and mixtures thereof. Preferred antioxidants include BHA, BHT, citric acid, and propyl gallate, and mixtures thereof. The antioxidants are generally added to the compositions in amounts of from about 0.01% w/w to about 2% w/w, based on total weight of the soft chew. More typically, the antioxidant(s) is generally at a concentration of about 0.01% ww to about 1% w/w, based on total weight of the soft chew.

The palatable soft chew composition further comprises at least one additional excipient. Non-exclusive examples of excipients include: sodium chloride, lactose, magnesium stearate, talc, meglumine, fumed silica, silicon dioxide, magnesium carbonate, colorants (e.g., natural and synthetics), stearin, stearic acid, alginate, calcium stearate, corn syrup, wheat germ, calcium phosphate dibasic anhydrous, confectionary sugar, sodium stearyl fumarate, and the like.

The palatable soft chew compositions comprising a compound of Formula (A1), (A2), (A3), or (A4), alone or in combination with at least one additional antiparasitic agent, are useful as a parasiticide for the control and treatment of parasitic infections and infestations in an animal. The veterinary compositions of the present invention have utility as an ectoparasiticide, in particular, as an acaricide and insecticide. They may, in particular, be used in the fields of veterinary medicine and the maintenance of public health: against acarines and insects which are parasitic upon animals. Some non-limiting examples of acarine and insect parasites include: ticks (e.g., Ixodes spp., Rhipicephalus spp., Boophilus spp., Amblyomma spp., Hyalomma spp., Haemaphysalis spp., Dermacentor spp., Ornithodorus spp., and the like); mites (e.g., Dermanyssus spp., Sarcoptes spp., Psoroptes spp., Eutrombicula spp., Chorioptes spp., Demodex spp., and the like); chewing and sucking lice (e.g., Damalinia spp., Linognathus spp., and the like); fleas (e.g., Siphonaptera spp., Ctenocephalides spp., and the like); and flies, mosquitos, and midges (e.g., Order Diptera; Aedes spp., Anopheles spp., Tabanidae spp., Haematobia spp., Stomoxys spp., Dermatobia spp., Simuliidae spp., Ceratopogonidae spp., Psychodidae spp., Cochliomyia spp., Muscidae spp., Hypoderma spp., Gastrophilus spp., Simulium spp., and the like. The compositions of the invention also have utility as an endoparaciticide, in particular, against nematodes, cestodes, and trematodes which are parasitic upon animals. Non-limiting examples of endoparasites include: Fasciola spp.; Fascioloides spp.; Paramphistomum spp.; Dicrocoelium spp.; Eurytrema spp.; Ophisthorchis spp.; Fasciolopsis spp.; Echinostoma spp.; Paragonimus spp.) and the phylum Nematoda (such as filarial, intestinal and tissue nematodes; e.g. Haemonchus spp.; Ostertagia spp.; Cooperia spp.; Oesphagastomum spp.; Nematodirus spp.; Dictyocaulus spp.; Trichuris spp.; Toxocara spp.; Toxascaris spp.; Trichinella spp.; Dirofilaria spp. (e.g., D. immitis); Ancyclostoma spp.; Necator spp.; Strongyloides spp.; Capillaria spp.; Ascaris spp.; Enterobius spp.; and Trichostrongylus spp.). Therefore, the veterinary compositions of the invention are of particular value in the control of ectoparasites, insects, and endoparasites which are injurious to, or spread or act as vectors of diseases in animals, for which purpose they may be administered orally.

The palatable soft chew compositions comprising a compound of Formula (B), carprofen, is useful for treating and preventing pain and inflammation in an animal, and more preferably in companion animals, particularly dogs. As is well known by skilled artisans (e.g., veterinarians), the canine species, i.e., dogs, especially older dogs, are very susceptible to chronic inflammatory processes such as degenerative joint disease. Carprofen is known to treat and prevent inflammatory processes and diseases in dogs by inhibiting the activity of inducible cyclo-oxygenase-2 (COX-2). Carprofen is a non-steroidal anti-inflammatory agent (NSAID). It has been used to treat and prevent inflammation and pain associated with arthritic symptoms. The inflammatory process itself may have a number of precipitating causes, including infectious agents, ischemia, antigen-antibody interactions, and thermal or other physical injury. The response to each of these causes is characteristically different, but they all have a strong commonality. Clinical symptoms include erythema, edema, tenderness and pain. Three distinct phases can be recognized, but each of these is mediated by different mechanisms. The first, acute transient phase involves local vasodilation and increased capillary permeability; the second, delayed, subacute phase involves infiltration of leukocytes and phagocytic cells; and the third, chronic proliferative phase involves tissue degeneration and fibrosis. NSAIDs as a therapeutic class of anti-inflammatory agents appear to act by inhibiting the enzymatic production and release of prostaglandins, which participate in the pathogenesis of inflammation and fever. In addition to anti-inflammatory actions, NSAIDs have analgesic, antipyretic, and platelet-inhibitory actions. They act by blocking the synthesis of prostaglandins by inhibiting cyclooxygenase, which converts arachidonic acid to cyclic endoperoxides, precursors of prostaglandins. Inhibition of prostaglandin synthesis accounts for their analgesic, antipyretic, and platelet-inhibitory actions; other mechanisms may contribute to their anti-inflammatory effects. Therefore, the veterinary compositions of the invention are of particular value in the treatment and prevention of pain and inflammation in animals, particularly dogs, for which purpose they may be administered orally.

The palatable soft chew compositions comprising a compound of Formula (C), oclacitinib (apoquel, the maleate salt of oclacitinib), is useful for modulating protein kinases, particularly janus kinases (JAK-1 and JAK-3). Protein kinases are families of enzymes that catalyze the phosphorylation of specific residues in proteins, broadly classified into tyrosine and serine/threonine kinases. Inappropriate kinase activity, arising from mutation, over-expression, or inappropriate regulation, dys-regulation or de-regulation, as well as over-or-under-production of growth factors or cytokines has been implicated in many diseases, including but not limited to cancer, cardiovascular diseases, allergies, asthma and other respiratory diseases, autoimmune diseases, inflammatory diseases, bone diseases, metabolic disorders, and neurological and neurodegenerative disorders. In addition, the JAK inhibitor, oclacitinib, is used to treat and prevent atopic dermatitis in animals. Inappropriate kinase activity triggers a variety of biological cellular responses relating to cell growth, cell differentiation, survival, apoptosis, mitogenesis, cell cycle control, and cell mobility implicated in the aforementioned and related diseases.

JAK-3 in particular, has been implicated in a variety of biological processes. For example, the proliferation and survival of murine mast cells induced by IL-4 and IL-9 have been shown to be dependent on JAK-3 and gamma chain-signaling (Suzuki et al., (2000), Blood 96:2172-2180). JAK-3 also plays a crucial role in IgE receptor-mediated mast cell degranulation responses (Malaviya et al., (1999), Biochem. Biophys. Res. Commun. 257:807-813). JAK-3 kinases have also been implicated in the mechanism involved in early and late stages of rheumatoid arthritis (Muller-Ladner et al., (2000), J. Immunal. 164:3894-3901. JAK-3 in particular plays an essential role in the function of lymphocytes, macrophages, and mast cells. Given the importance of this JAK kinase, compounds which modulate the JAK pathway, including those selective for JAK-3, can be useful for treating diseases or conditions where the function of lymphocytes, macrophages, or mast cells is involved (Kudlacz et al., (2004) Am. J. Transplant 4:51-57; Changelian (2003) Science 302:875-878). Conditions in which targeting of the JAK pathway or modulation of the JAK kinases, particularly JAK-3, are contemplated to be therapeutically useful include, arthritis, asthma, autoimmune diseases, cancers or tumors, diabetes, certain eye diseases, disorders or conditions, inflammation, intestinal inflammations, allergies or conditions, neurodegenerative diseases, psoriasis, transplant rejection, and viral infection. Oclacitinib inhibits the function of a variety of pruritogenic cytokines and pro-inflammatory cytokines, as well as cytokines involved in allergy that are dependent on JAK-1 or JAK-3. In addition, oclacitinib can be used for the treatment and prevention of skin diseases, conditions or disorders including atopic dermatitis, eczema, psoriasis, scleroderma, pruritus and other pruritic conditions; allergic reactions including allergic dermatitis in animals, including horse allergic diseases such as bite hypersensitivity, summer eczema and sweet itch in horses.

The palatable soft chew compositions comprising a compound of Formula (D), a neurokinin (NK)-1 inhibitor, is useful for treating emesis in an animal, particularly, dogs and cats. The treatment of emesis includes the treatment of nausea, retching and vomiting. Emesis includes acute emesis, delayed emesis and anticipatory emesis. Substance P receptor antagonists are useful in the treatment of emesis, however induced. For example, emesis may be induced by drugs such as cancer chemotherapeutic agents (e.g., cyclophosphamide, carmustine, lomustine and chlorambucil), cytotoxic antibiotics (e.g., dactinomycin, doxorubicin, mitomycin-C and bleomycin), opioid analgesics (e.g., morphine), anti-metabolites (e.g., cytarabine, methotrexate and 5-fluorouracil), vinca alkaloids (e.g., etoposide, vinblastine and vincristine), and other drugs such as cisplatin, dacarbazine, procarbazine and hydroxyurea. Emesis may also be induced by radiation sickness, radiation therapy, poisons, toxins such as those caused by metabolic disorders or by infection (e.g., gastritis), pregnancy, vestibular disorders such as motion sickness, post-operative sickness, gastrointestinal obstruction, reduced gastrointestinal motility, visceral pain (e.g., myocardial infarction or peritonitis), migraine, increased intracranial pressure or decreased intracranial pressure (e.g., altitude sickness). The palatable soft chew composition comprising maropitant can also be used for managing pain, and in particular, visceral pain in animals.

The palatable soft chew compositions comprising an antibiotic are useful for treating a microbial infection in animals, particularly dogs and cats.

It is to be understood by one of ordinary skill in the art that the present discussion is a description of exemplary embodiments only and is not intended as limiting the broader aspects of the present invention, which broader aspects are embodied in the exemplary construction. In fact, it will be apparent to those skilled in the art that various modifications and variations can be made in the present invention without departing from the scope or spirit of the invention. For instance, features illustrated or described as part of one embodiment can be used in another embodiment to yield a still further embodiment. It is intended that the present invention cover such modifications and variations as come within the scope of the appended claims and their equivalents.

The palatable, soft chew composition of the invention can be prepared using a two-step process. First, the powdered (dry) ingredients are mixed together and then combined with at least one wetting agent in an extruder to form the extrudate. At the die end of the extruder, the extrudate is passed through a conditioning chamber having a temperature range of about −10° C. to about 8° C. for about 1 to about 5 minutes, preferably about 2 minutes for drying. The dried soft chew strands can then be cut to similar size and dimension. Alternatively, the soft chew strands can be air dried or placed into a tray drier at a temperature of about 40 to 60° C., preferably about 50° C. for about 24 hours before cutting to similar size and dimension. Once cut, the soft chew tablets can be packaged. The extruder used for making the soft chew strands can be a single screw extruder or a twin screw extruder. Twin screw extruders are preferred for better mixing. The liquid can also be introduced directly into the extruder through a barrel port. An example of a suitable commercial extruder is the TX-85 twin screw extruder from Wenger.

### EXAMPLES

The invention is further described by the following non-limiting examples which further illustrate the invention, and are not intended, nor should they be interpreted to, limit the scope of the invention.

Palatable soft-chew compositions containing carprofen were prepared and evaluated for palatability. Example 1 was prepared by accurately weighing out each ingredient and active agent. The dry components were added together and mixed. The homogenous blend was transferred from the blender to an extruder hopper. A wetting agent, for example, glycerol, was pumped into the extruder through an extruder port. The dry blend and glycerol were subsequently mixed by the extrusion screw, and the extrudate collected, dried, and cut. Subsequent non-limiting examples can be similarly prepared.

### Example 1

| Excipient/Active | w/w % |
|---|---|
| Carprofen | 5 |
| Microcrystalline cellulose | 15 |
| Disintegrant | 5 |
| antioxidant | 0.02 |
| Sodium chloride | 0.1 |
| glycerol | 28 |
| Flavorant/filler | 45 |
| PVP K-30 | 2 |

### Example 2

| Excipient/Active | w/w % |
|---|---|
| Carprofen | 2 |
| Microcrystalline cellulose | 15 |
| Disintegrant | 10 |
| glycerol | 30 |
| Flavorant/filler | 41 |
| PVP K-30 | 2 |

### Example 3

| Excipient/Active | w/w % |
|---|---|
| Carprofen | 3.75 |
| Microcrystalline cellulose | 15 |
| Disintegrant | 5 |
| antioxidant | 0.02 |
| Sodium chloride | 0.1 |
| glycerol | 28 |
| Flavorant/filler | 46.25 |
| PVP K-30 | 2 |

### Example 4

| Excipient/Active | w/w % |
|---|---|
| Carprofen | 2 |
| Microcrystalline cellulose | 10 |
| Disintegrant | 12 |
| glycerol | 31 |
| Flavorant/filler | 43 |
| PVP K-30 | 2 |

### Example 5

| Excipient/Active | w/w % |
|---|---|
| Carprofen | 4 |
| microcrystalline cellulose | 10 |
| Disintegrant | 12 |
| glycerol | 29 |
| Flavorant/filler | 43 |
| PVP K-30 | 2 |

### Example 6

| Excipient/Active | w/w % |
|---|---|
| Carprofen | 5 |
| microcrystalline cellulose | 15 |
| Antioxidant | 0.2 |
| Sodium chloride | 0.1 |
| Color | 0.1 |
| Talc/magnesium stearate | 3 |
| Disintegrant | 5 |
| glycerol | 24.6 |
| Flavorant/filler | 43 |
| PVP K-30 | 4 |

### Example 7

| Excipient/Active | w/w % |
|---|---|
| Carprofen | 2.5 |
| microcrystalline cellulose | 15 |
| Antioxidant | 0.2 |
| Sodium chloride | 0.1 |
| Color | 0.1 |
| Magnesium stearate | 3 |
| Disintegrant | 5 |
| glycerol | 24.6 |
| Flavorant/filler | 45.5 |
| PVP K-30 | 4 |

### Example 8

| Excipient/Active | w/w % |
|---|---|
| Carprofen | 2.5 |
| microcrystalline cellulose | 15 |
| Antioxidant | 0.2 |
| Sodium chloride | 0.1 |
| Color | 0.1 |
| Talc/Magnesium stearate | 3 |
| meglumine | 2.5 |
| Disintegrant | 5 |
| glycerol | 24.6 |
| Flavorant/filler | 43 |
| PVP K-30 | 4 |

### Example 9

| Excipient/Active | w/w % |
|---|---|
| Carprofen | 5 |
| microcrystalline cellulose | 15 |
| Antioxidant | 0.2 |
| Sodium chloride | 0.1 |
| Color | 0.1 |
| Talc/magnesium stearate | 3 |
| meglumine | 2.5 |
| Disintegrant | 5 |
| glycerol | 24.6 |
| Flavorant/filler | 40.5 |
| PVP K-30 | 4 |

### Example 10

| Excipient/Active | w/w % |
|---|---|
| Maropitant | 2.5 |
| Microcrystalline cellulose | 15 |
| Disintegrant | 10 |
| glycerol | 28 |
| Flavorant/filler | 40.5 |
| PVP K-30 | 4 |

### Example 11

| Excipient/Active | w/w % |
|---|---|
| Apoquel | 1.6 |
| Microcrystalline cellulose | 15 |
| Disintegrant | 10 |
| glycerol | 28 |
| Flavorant/filler | 43.4 |
| PVP K-30 | 2 |

### Example 12

| Excipient/Active | w/w % |
|---|---|
| Sarolaner | 1 |
| Microcrystalline cellulose | 15 |
| Disintegrant | 10 |
| glycerol | 28 |
| Flavorant/filler | 44 |
| PVP K-30 | 2 |

### Biological

Two separate cross-over studies were conducted in beagle dogs to assess composition palatability and overall acceptance. In each study, dogs (8/group and 12/group) were offered a single dose once daily for three days. In Study 1, 16 dogs received either a 100 mg carprofen soft chew tablet (Example 1) or soft chew placebo tablet. Overall acceptance based on consumption was about 94.4% for placebo and 95.8% for carprofen. In the second study, 24 dogs received either carprofen (100 mg, Example 1) soft chew tablet or Example 1 minus the microcrystalline cellulose (adjusted with flavorant/filler). Overall acceptance of the carprofen soft chew Example 1 and Example 1 without microcrystalline cellulose was 94.4% and 90.3%, respectively. Overall, palatability and overall consumption of the carprofen soft-chews was equal to or better than that observed for the placebo soft chew and the addition of microcrystalline cellulose increased the tablet acceptance.

The stability of Example 1 at 1-month 40° C./75% relative humidity was 98.6%; at 2-months at 40° C./75% relative humidity was 99.1%; and at 2-months 25° C./60% relative humidity was 98.2%.

We claim:

1. A palatable soft chew veterinary composition comprising:
1. a therapeutically effective amount of at least one active agent;
2. at least one binding agent;
3. at least one disintegrant,
4. at least one wetting agent, and
5. at least one flavorant.

2. The composition of claim 1, wherein the binding agent is selected from the group consisting of microcrystalline cellulose, hydroxypropyl cellulose, polyvinylpyrrolidone, and mixtures thereof and wherein the binding agent makes up about 10 to about 25 w/w % of the composition.

3. The composition of claim 1, wherein the wetting agent is selected from the group consisting of glycerin, glycerol formal, ethylene glycol, propylene glycol, butyl diglycol, castor oil, cottonseed oil, sesame oil, safflower oil, soybean oil, corn oil, glyceryl stearates, isopropyl myristate, ethyl oleate, and propylene glycol monolaurate, and mixtures thereof, and wherein the wetting agent makes up about 20 to about 35 w/w % of the composition.

4. The composition of claim 3 wherein said disintegrant is croscarmellose sodium and makes up about 3 to about 15 w/w % of the composition.

5. The composition of claim 4 wherein said flavorant is an animal based natural flavorant, artificial flavorant, or mixture thereof and wherein the composition further comprises an antioxidant.

6. The composition of claim 1 wherein the active agent is selected from carprofen, oclacitinib maleate, afoxolaner, fluralaner, sarolaner, lotilaner, clavulanic acid and amoxicillin, cefpodoxime proxetil, and doxycycline hyclate.

7. The composition of claim 6 wherein the active agent is selected from afoxolaner, fluralaner, sarolaner, and lotilaner, and wherein said composition further comprises at least one additional antiparasitic agent.

8. The composition of claim 7 wherein the at least one additional antiparasitic agent is selected from the group consisting of a macrocyclic lactone, pyrantel, praziquantel, levamisole, triclabendazole, and mixtures thereof.

9. A method of treating an animal for a parasitic infection, pain and inflammation, pruritis and atopic dermatitis, or emesis by orally administering a palatable soft chew composition comprising
a. a therapeutically effective amount of at least one active agent;
b. at least one binding agent;
c. at least one disintegrant,
d. at least one wetting agent, and
e. at least one flavorant; and optionally
f. at least one additional antiparasitic agent.

10. The method of claim 9 for treating an animal for pain and inflammation by orally administering a palatable soft chew composition comprising
a. a therapeutically effective amount of at least one active agent, wherein said agent is carprofen;
b. at least one binding agent;
c. at least one disintegrant,
d. at least one wetting agent, and
e. at least one flavorant.

11. The method of claim 9 for treating an animal for pruritis and atopic dermatitis infection by orally administering a palatable soft chew composition comprising
a. a therapeutically effective amount of at least one active agent, wherein said agent is apoquel;
b. at least one binding agent;
c. at least one disintegrant,
d. at least one wetting agent, and
e. at least one flavorant.

12. The method of claim 9 for treating an animal for emesis by orally administering a palatable soft chew composition comprising
a. a therapeutically effective amount of at least one active agent, wherein said agent is maropitant;
b. at least one binding agent;
c. at least one disintegrant,
d. at least one wetting agent, and
e. at least one flavorant.

13. The method of claim 9 wherein the animal is a companion animal.

14. The method of claim 13 wherein the companion animal is a dog.

15. A process for preparing a palatable soft chew veterinary composition of claim 1, comprising:
1. sieving and blending all dry ingredients;
2. loading the blended dry ingredients into an extruder hopper;
3. wetting the blended ingredients in the extruder at a temperature range of about 20-30° C.;
4. drying and cutting the extrudate.

16. The composition of claim 6 wherein the active agent is carprofen.

17. The composition of claim 6 wherein the active agent is oclacitinib maleate.

18. The composition of claim 6 wherein the active agent is clavulanic acid and amoxicillin.

19. The method of claim 9 for treating an animal for a parasitic infection by orally administering a palatable soft chew composition comprising
a. a therapeutically effective amount of at least one active agent selected from afoxolaner, fluralaner, lotilaner, and sarolaner;
b. at least one binding agent;
c. at least one disintegrant,
d. at least one wetting agent, and
e. at least one flavorant; and optionally
f. at least one additional antiparasitic agent.

20. The method of claim 19 wherein the active agent is sarolaner.
